(19) United States
(12) Patent Application Publication
(10) Pub. No.: US 2009/0038316 A1
(43) Pub. Date: Feb. 12, 2009

(54) INTEGRATED PROCESS FOR CARBONACEOUS MATERIAL TO CO2-FREE FUEL GAS FOR POWER PLANTS AND TO ETHYLENE

(75) Inventor: Stanley R. Pearson, Baton Rouge, LA (US)

Correspondence Address: KEAN, MILLER, HAWTHORNE, D'ARMOND,;MCCOWAN & JARMAN, L.L.P., ONE AMERICAN PLACE, 22ND FLOOR, P.O. BOX 3513, BATON ROUGE, LA, 70821, US

(73) Assignee: International Financial Services #1, LLC, Baton Rouge, LA (US)

(21) Appl. No.: 12/188,872

(22) Filed: Aug. 8, 2008

Related U.S. Application Data
(60) Provisional application No. 60/955,243, filed on Aug. 10, 2007.

(51) Int. Cl. F02C 3/20; F02C 3/34; C10J 3/00; C10K 1/34; C10K 3/00; C07C 27/00

(52) U.S. Cl. 60/784; 60/39.12; 48/198.3; 48/202; 423/220; 518/700

(57) ABSTRACT

An integrated process wherein a CO₂-free fuel gas is produced from steam reforming a carbonaceous material and sent to a gas turbine which is associated with an electrical generator. The CO₂ produced during the steam reforming of the carbonaceous material is passed to a second steam reforming zone where it is mixed with a second carbonaceous feed to produce a syn-gas that is used to produce ethylene.

### CROSS REFERENCE TO RELATED APPLICATIONS

This application is based on Provisional Application 60/955,243 filed Aug. 10, 2007.

### FIELD OF THE INVENTION

This invention relates to an integrated process wherein a CO₂-free fuel gas is produced from steam reforming a carbonaceous material and sent to a gas turbine which is associated with an electrical generator. The CO₂ produced during the steam reforming of the carbonaceous material is passed to a second steam reforming zone where it is mixed with a second carbonaceous feed to produce a syn-gas that is used to produce ethylene.

### BACKGROUND OF THE INVENTION

Coal-fired power plants are the largest source of air pollution in the United States. When coal is burned, pollution comes out of the smokestacks and is released into the atmosphere. Some such pollutants are nitrogen oxides, sulfur dioxide, carbon dioxide, mercury and various toxins. Also, power plant pollution can be linked to asthma attacks and other incidents of upper respiratory symptoms. Such health risks are greatest for people living within the vicinity of the plants.

Power plants emit 40% of total U.S. carbon dioxide pollution—the primary global warming pollutant. Although coal-fired power plants account for just over half of the electricity produced in the U.S. each year, they have been responsible for over 83% of the CO₂ pollution since 1990. Coal-fired power plants have the highest output rate of CO₂ per unit of electricity compared to other fossil fuels.

Much work has gone to generate electrical power without direct combustion of coal. For example, U.S. Pat. No. 4,566,267 teaches a power generating plant with an integrated coal gasification plant and an ammonia synthesis plant in which not only is heat extracted from the raw gas from the coal gasifier and utilized to generate steam which is used in the steam turbine of the steam power generating plant but a substantial portion of the raw gas after treatment is converted to ammonia in an ammonia synthesis plant.

While various technologies have been used in an attempt to produce clean energy from coal there is still a need for better and more efficient and less polluting power plants that use coal as a primary fuel source.

### SUMMARY OF THE INVENTION

In accordance with the present invention there is provided a process for generating electrical power from a coal fuel source while producing low carbon number alcohols, which process comprises:

i) introducing a first carbonaceous feedstock and an effective amount of steam into a first reforming zone operated under reforming conditions thereby producing a fuel gas product stream comprised of solids, H₂, CO, CH₄, CO₂ and H₂S, which fuel gas product stream is a high temperature stream;

ii) passing said high temperature fuel gas stream to a heat recovery zone wherein its temperature is reduced to a temperature suitable for a CO-shift conversion reaction zone and wherein at least a portion of the heat of the fuel gas is utilized to generate steam;

iii) passing said fuel gas stream which is now at a lower temperature to a solids recovery zone wherein a substantial amount of the solids of said lower temperature fuel gas stream are removed, thereby resulting in a substantially solids-free lower temperature fuel gas stream;

iv) conducting said substantially solids-free fuel gas stream to a CO shift conversion zone operated at a temperature from about 180° C. to about 280° C. wherein CO is reacted with H₂O in the presence of a shift conversion catalyst to covert at least a portion of the CO and H₂O into CO₂ and H₂, thereby resulting in a substantially solids-free CO-lean fuel gas stream comprised primarily of CO₂, H₂S, CH₄ and H₂;

v) conducting said substantially solids-free fuel gas stream resulting from step iv) to a heat recovery zone wherein the stream is reduced to a temperature effective for conducting to an acid scrubbing zone;

vi) conducting said substantially solids-free fuel gas steam of step v) to an acid gas scrubbing zone wherein at least a portion of the H₂S and CO₂ are removed, thereby resulting in an acid gas rich stream and an acid gas lean fuel gas stream, which substantially acid gas lean fuel gas stream contains at least about 80 vol. % H₂;

vii) conducting said substantially acid gas lean fuel gas stream from said acid gas scrubbing zone to a power plant wherein it is used as fuel to a gas turbine associated with an electrical generator;

viii) conducting said acid gas rich stream to a sulfur removal zone wherein sulfur compounds, including H₂S, are removed thereby resulting in a CO₂-rich stream;

ix) conducting said CO₂-rich stream along with a second carbonaceous feedstock to a second reforming zone operated under reforming conditions including temperatures from about 650° F. to about 1750° F. wherein a syn-gas product stream is produced comprised of solids, H₂, CO, CH₄, and CO₂;

x) passing said syn-gas product stream to a second heat recovery zone wherein its temperature is reduced and wherein at least a portion of the heat of the syn-gas is utilized to generate steam;

xi) passing said syn-gas stream now at a lower temperature to a solids recovery zone wherein a substantial amount of the solids of the solids waste stream are removed thereby resulting in a substantially solids-free lower temperature syn-gas stream; and

xii) passing said substantially solids-free lower temperature syn-gas stream to a second acid gas removal zone wherein substantially all of the CO₂ is removed, thereby resulting in an acid gas rich stream and an acid gas lean syn-gas stream comprised primarily of H₂, CH₄ and CO;

xiii) passing at least a portion of said acid gas lean syn-gas stream to a Fischer-Tropsch reaction unit containing a suitable catalyst for the production of methanol and operated at Fischer-Tropsch reaction conditions, thereby producing a stream containing predominantly methanol;

xiv) passing at least a portion of said methanol and a portion of said lean syn-gas stream of step xiii) above to a Fischer-Tropsch reaction unit containing a suitable catalyst for the production of ethanol and operated at Fischer-Tropsch reaction conditions, thereby producing a stream containing predominantly ethanol; and

xv) conducting at least a portion of the ethanol to a dehydration zone where at least a portion of the ethanol is converted to ethylene.

In a preferred embodiment said first reforming zone is comprised of three temperature zones, each serially and fluidly connected to each other and each at a higher temperature than the previous immediate upstream temperature zone which respect to the flow of feedstock.

In another preferred embodiment the first carbonaceous feedstock is a coal selected from lignite, sub-bituminous, bituminous, and anthracite.

In yet another preferred embodiment said first and second acid gas scrubbing zones contains an amine solution.

In still another preferred embodiment the acid gas lean stream of step vi) contains at least about 90 vol. % H₂.

### BRIEF DESCRIPTION OF THE FIGURE

FIG. 1 hereof is a generalized flow scheme of a preferred embodiment of the present invention showing the integration of a coal gasification process unit, a power plant run on a fuel gas generated by the coal gasification process unit, and an alcohol unit wherein a CO₂ stream generated in the coal gasification process unit is a co-feed with a biomass feed to produce ethanol which is then converted to ethylene.

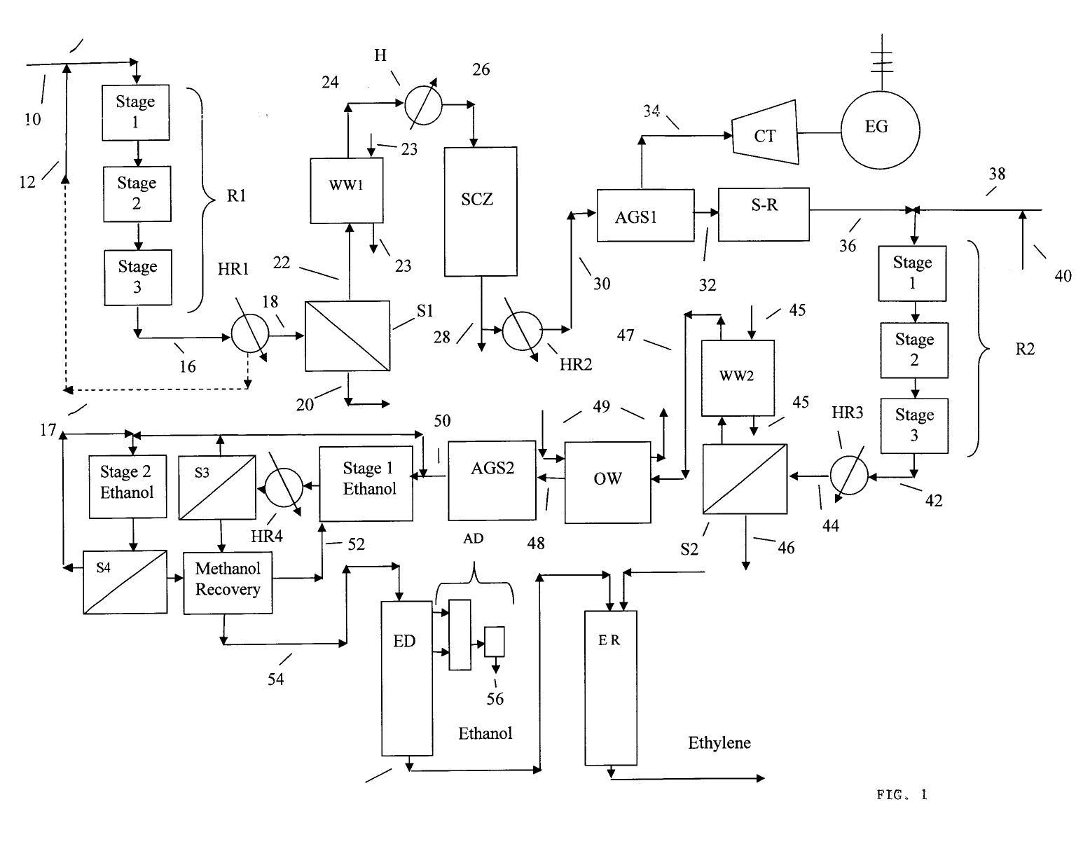

### DETAILED DESCRIPTION OF THE INVENTION

The present invention is primarily directed to a CO₂-free power plant for generating electricity. Any suitable carbonaceous material (solid, liquid or gaseous) that is capable of being used as a fuel source can be used in the practice of the present invention. Non-limiting examples of such carbonaceous materials that can be used in the practice of the present invention include: i) petroleum derived carbonaceous materials such as methane, heavy hydrocarbonaceous oils, heavy and reduced petroleum crude oils, petroleum atmospheric bottoms, petroleum vacuum distillation bottoms, heavy hydrocarbon residues and asphalt; ii) bitumens, tar sand oil, pitch, and shale oil; iii) natural gas; iv) coal; v) coal derived materials including coals such as liqnite, sub-bituminous, bituminous, and antrhracite and coal liquid products obtained from coal liquefaction as well as gaseous products obtained by coal gasification; and vi) biomass feeds.

The term “biomass” as used herein is intended to refer to any non-fossilized, i.e., renewable organic matter collected for use as a source of energy. The various types of biomass include plant biomass (defined below), animal biomass (any animal by-product, animal waste, etc.) and municipal waste biomass (residential and light commercial refuse with recyclables, such as metal and glass removed). The term “plant biomass” or “lingo-cellulosic biomass” as used herein is intended to refer to virtually any plant-derived organic matter (woody and non-woody) available for energy on a sustainable basis. Plant biomass can include, but is not limited to, agricultural crops, such as corn, and agricultural crop wastes and residues such as corn stover, corn cobs, wheat straw, rice straw, rice hulls, kennaf, distillers grains, sugar can bagasse and the like. Plant biomass further includes, but in not limited to cellulosic based materials such as woody energy crops, wood wastes such as old railroad ties, and residues such as trees, softwood forest thinnings, barky wastes, sawdust, paper and pulp industry waste streams, wood fiber, and the like. Additionally grass crops, such as switch grass and the like have potential to be produced large-scale as another plant biomass source. For urban areas, the best potential plant biomass feedstock comprises yard waste (e.g., grass clippings, leaves, tree clippings, brush, etc.) and vegetable processing waste.

When a carbonaceous material, such as coal, is used as the primarily directed to a process wherein a coal feedstock is reformed to produce a fuel gas. The acid gases H₂S and CO₂ are removed from the fuel gas which is sent to a power plant as fuel, thereby resulting in a CO₂-free electrical power plant. The CO₂ from coal reforming is conducted, along with a suitable biomass to a second reforming zone wherein a syn-gas is produced and used to produce ethanol. Conventional power plant technology typically injects the CO₂ into the ground, or disposes of it in some other less preferred method of disposal. The practice of the present invention utilizes the CO₂ produced from coal reforming to produce low carbon number alcohols, such as methanol and ethanol, which in turn can be used to produce ethylene, which can be fed to a chemicals plant.

Any type and rank of coal, other than graphite, can be used in the practice of the present invention. Non-limiting examples of such coals include lignite, sub-bituminous, bituminous and anthracite. Lignite, which is also referred to as brown coal, is the lowest rank of coal and is used almost exclusively as fuel for steam-electric power generation. Sub-bituminous coal, whose properties range from those of lignite to those of bituminous coal is used primarily as fuel for steam-electric power generation. Bituminous coal is a relatively dense coal and is also used primarily as fuel in steam-electric power generation, with substantial quantities also used for heat and power applications in manufacturing and to make fuel grade coke. Anthracite is the highest rank coal and is a harder, glossy, black coal that is used primarily for residential and commercial space heating, although it can also be used in the practice of the present invention.

This invention can be better understood with reference to the sole FIGURE hereof. Coal, in a pulverized form, is conducted via line 10 and superheated steam which is conducted via line 12 to first reforming zone R1. The size of the coal particles will preferably be from about 1/32 inch to about 1 inch, preferably from about 3/16 inch to about ½ inch. Any suitable technique can be used to reduce the particle size of the coal to the required size. The superheated steam, which will be at a temperature from about 850° F. to about 950° F. acts as both a source of hydrogen as well as a transport medium. When mixed with the coal the resulting mixture must be kept above its dew point before entering the reforming zone. The dew point will typically be at about 230° C. The amount of superheated steam to coal feedstock will be an effective amount. By effective amount we mean at least that amount needed to provide sufficient transport of the coal. That ratio of superheated to steam of coal, on a volume to volume basis will typically be from about 0.2 to 2.5, preferably from about 0.3 to 1.0. The steam is preferably introduced so that the coal is diluted to the point where it can easily be transported through the reactor tubes of the reformer. Fluidization will typically result and can realize fluid reforming by virtue of good contact among steam, polymers and heat decomposition products of carbonaceous material liberated in the gas phase.

The mixture of steam and coal feedstock is fed to first reforming zone R1 via line 14 where it is converted into a fuel gas, also referred to herein as a fuel gas. While any type of reforming process unit can be used in the practice of the present invention so long as it is capable of converting coal to a fuel gas, it is preferred that the reforming zone be comprised of at least three temperature stages. It was found by the inventor hereof that the use of three temperature stages, each of a progressively higher temperature than the previous upstream stage will result in a fuel gas having a substantially higher hydrogen content than other more conventional reforming process units. The coal and superheated steam are conducted into stage 1 of reforming zone R1 which is operated at a temperature of about 650° F. (343° C.) to about 800° F. (426° C.). The lower boiling volatiles will be driven off in this stage. The remaining coal, steam and lower boiling volatiles will then pass to stage 2 which is operated in the temperature range of about 800° F. (426° C.) to about 1400° F. (760° C.) and then to stage 3 which is operated in the temperature range of about 1400° F. (760° C.) to about 1750° F. (954° C.). While low to medium ranked coals, such as lignite to bituminous, may be processed in these three temperature stages it will be understood that a fourth temperature stage (not shown), operating at a temperature greater than about 1750° F., may be needed for a high ranked coal such as anthracite. The effluent from first reforming zone R1 will be solids, such as ash, water vapor, and a syn-gas comprised primarily of CO, CO₂, H₂S, CH₄ and H₂. Each stage of first reforming zone R1 will be comprised of a plurality of straight or coiled reactor tubes of effective internal diameter and length within a metal vessel, preferably cylindrical, of suitable size. Typical internal diameters for the reactor tubes will be from about 2 to about 6 inches, preferably from about 2.5 to about 3.5 inches. It is also preferred, that each stage be a separate reactor vessels, although it is possible, but not preferred to have two or more temperature stages in a single reactor vessel.

Although the source of heat for the reforming zone can be any suitable source it is preferred that the source of heat be one or more burners (not shown) located at the bottom each reactor of each stage, except for stage 1. The fuel for the one or more burners can be any suitable fuel. It is preferred that at least some of the fuel be obtained from the process itself, such as the fuel gas produced in the one of the reforming zones.

The inlet temperature of the feedstock and superheated steam entering both reforming zones R1 and R2 will preferably be about 230° C. The exit temperature of the product fuel gas exiting each reforming zone, via line 16 for R1 and line 42 for R2 will typically be at a temperature of about 1600° F. to about 2000° F. At a temperature of about 1100° C. and above and with a contact time of about 1 second, one obtains less than about one mole percent of methane and about 1 mol % CO, which is the desirable result. Pressure of each reformer is not critical, but it will typically be at about 3 to 350 psig. Also, it is preferred that the residence time in each reformer be from about 0.4 to about 1.5 seconds.

For any given feedstock, one can vary the proportions of hydrogen, carbon dioxide, carbon monoxide and methane that comprise the resulting fuel gas product stream by adjusting such things as contact time of the feedstock in the reformer, the exit temperature, the amount of steam introduced, and to a lesser extent the pressure. Certain proportions of fuel components are better than others for producing other products, thus conditions should be such as to maximize the production of hydrogen and methane at the expense of carbon oxides.

Returning now to the FIGURE, the product fuel gas stream from first reforming zone R1 is conducted via line 16 to first heat recovery zone HR1 where it is preferred that water be the heat exchange medium and that the water be used as preheated steam for stage 1 via line 17. First heat recovery zone HR1, as well as second heat recovery zone HR2 (discussed later), can be any suitable heat exchange device, such as a shell-and-tube type wherein water is used to remove heat from product stream 16. Such heat recovery devices are often called waste heat boilers. From heat recovery zone HR1 the product fuel gas is passed via line 18 through first separation zone S which contains a gas filtering means and preferably a cyclone (not shown) and optionally a bag house (not shown) to remove at least a portion, preferably substantially all, of the remaining ash and other solid fines from the fuel gas. The filtered solids, such as ash, are collected via line 20 for disposal.

The filtered fuel gas stream is then passed via line 22 to first water wash zone WW1 wherein it is conducted upward and countercurrent to down-flowing water via line 23. The water wash zone preferably comprises a column packed with conventional packing material, such as copper tubing, pall rings, metal mesh or other such materials. The fuel gas passes upward countercurrent to down-flowing water which also serves to further cool the fuel gas stream, preferably to a temperature needed for the CO-shift conversion reaction zone. It also removes any remaining ash that may not have been removed in first separation zone S1. If the water washed fuel gas stream is at a temperature lower than needed for the CO-shift conversion reaction zone it can be passed via line 24 through optional heater H to bring it to reaction temperature. CO and H₂O are converted to CO₂ and H₂ in CO-shift conversion zone SCZ. It will be understood that water in excess of a stoichiometric amount needed for the shift reaction is removed before the fuel gas stream is introduced into shift conversion zone SCZ. The heated fuel gas stream is conducted into the CO shift conversion zone SCZ which contains a suitable shift conversion catalyst, preferably one containing of cobalt and molybdenum sulfides. Such catalysts are readily available from suppliers such as Johnson Matthey.

The exit gas from shift converter zone SCZ is then sent via line 28 to second heat recovery zone HR2 where it is cooled to a temperature of about 100° F. (37° C.) to about 110° F. (43° C.) and sent via line 30 to first acid gas scrubbing zone AGS1. Any suitable acid gas scrubbing technology can be used in the practice of the present invention. One suitable acid gas scrubbing technology is the use of an amine scrubber. Non-limiting examples of such basic solutions are the amines, preferably diethanol amine, mono-ethanol amine, and the like. More preferred is diethanol amine. Another preferred acid gas scrubbing technology is the so-called “Rectisol Wash” which uses an organic solvent, typically methanol, at subzero temperatures. Selexol and Purisol are also suitable acid gas scrubbing technologies. The scrubbed stream can also be passed through one or more guard beds (not shown) to remove catalyst poisoning impurities such as sulfur, halides etc. The ratio of H₂S to CO₂ of the fuel gas entering the acid gas scrubbing zone will depend on the type of coal used as the feedstock to the reformer. For example, if the coal is a low sulfur coal then the ratio of H₂S to CO₂ may be too low for recovery in a downstream Claus plant. Claus plants are the most significant gas desulfurizing process, recovering sulfur from gaseous H₂S. Typically, the gas entering the Claus plant will be required to have at least about 25 vol. % H₂S. Thus, for coals having a low sulfur level, the level of H₂S may be too low to be sent directly to a Claus plant. In such cases, an effective amount of CO₂ is removed, preferably by use of a suitable absorption bed, from the stream to increase the concentration of H₂S with respect to CO₂ to acceptable levels for a Claus plant. CO₂ absorbers are well known in the art.

A gaseous stream containing at least about 25 vol. % H₂S is sent to a sulfur recovery zone S—R via line 32. The preferred sulfur recovery zone S—R is a Claus plant, which is well known in the art. Another gaseous stream, one that is a substantially acid gas-free fuel gas stream containing at least about 80 vol. %, preferably at least 85 vol. %, more preferably at least about 90 vol. %, and most preferably at least about 92 vol. % hydrogen is passed via line 34 to combustion turbine CT to drive an electrical generator EG that produces power. A CO₂-rich stream exits sulfur recovery zone S—R via line 36 and is sent, along with a second carbonaceous feedstock, which can be a suitable biomass, via line 38 and superheated steam via line 40 to second reforming zone R2 to produce a sy gas product comprised primarily of CO, CO₂, CH₄ and H₂. Although any type of steam reformer can be used for converting biomass to a syn-gas, it is preferred that it be of the type described above for R1, that has a plurality of serially positioned temperature stages wherein the feed progresses from a first stage to a last stage at progressively higher temperatures. The temperature range for each stage of R2 will be as described above for R1.

Cellulosic materials are the more preferred biomass feedstocks, with wood being the most preferred. Biomass is typically comprised of three major components: cellulose, hemicellulose and lignin. Cellulose is a straight and relatively stiff molecule with a polymerization degree of approximately 10,000 glucose units (C₆ sugar). Hemicellulose are polymers built of C₅ and C₆ sugars with a polymerization degree of about 200 glucose units. Both cellulose and hemicellulose can be vaporized with negligible char formation at temperatures above about 500° C. On the other hand, lignin is a three dimensional branched polymer composed of phenolic units. Due to the aromatic content of lignin, it degrades slowly on heating and contributes to a major fraction of undesirable char formation. In addition to the major cell wall composition of cellulose, hemicellulose and lignin, biomass often contains varying amounts of species called “extractives”. These extractives, which are soluble in polar or non-polar solvents, are comprised of terpenes, fatty acids, aromatic compounds and volatile oil.

In most instances the biomass feedstock used in the practice of the present invention will be in a form of particles too large for transporting through the tubes of the reforming apparatus. Thus, it may be necessary to grind the biomass material to an effective size. In this case, the feedstocks are ground, or otherwise reduced in size, to a suitable size of about 1/32 inch to about 1 inch, preferably about 3/16 inch to about ½ inch. Grinding techniques are well know and varied, thus any suitable grinding technique and equipment can be used for the particular carbonaceous material being converted.

For any given biomass feedstock, one can vary the proportions of hydrogen, carbon dioxide, carbon monoxide and methane that comprise the resulting syn-gas product stream by adjusting such things as the contact time of the feedstock in the reforming zone, the exit temperature, the amount of steam introduced, and to a lesser extent, pressure. Certain proportions of syn-gas components are better than others for producing synthetic natural gas, thus conditions should be such as to maximize the production of methane and hydrogen.

The effluent syn-gas exiting second reforming zone R2 is passed via line 42 to third heat recover zone HR3 which has the same requirements as previously discussed for heat recovery zones HR1 and HR2. The cooled stream from third heat recovery zone HR3 is passed via line 44 to second separation zone S2 where at least a portion of the solids are removed via line 46 Second solids separation zone S2, like first solids separation zone S1, can include any suitable separation apparatus, such as cyclones, bag houses, filters and the like. The resulting solids-lean product syn-gas stream is conducted from second solids separation zone S2 to second water wash zone WW2 where it is conducted upward and countercurrent to down-flowing water via line 45. The water wash zone preferably comprises a column packed with conventional packing material, such as copper tubing, pall rings, metal mesh or other such materials. The syn-gas passes upward countercurrent to down-flowing water which serves to further cool the syn-gas stream to about ambient temperature. It also removes any remaining ash that may not have been removed in separation zone S2

The water washed syn-gas stream is then passed via line 47 to an oil wash zone OW where it is passed countercurrent to a down-flowing organic liquid via line 49 to remove any organics present, such as benzene, toluene, xylene, or heavier hydrocarbon components that may have been produced in the reformer. The down-flowing organic liquid will be any organic liquid in which the organic material being removed is substantially soluble. It is preferred that the down-flowing organic liquid be a petroleum fraction, such as one boiling in the naphtha to distillate boiling range, more preferably a C₁₆ to C₂₀ hydrocarbon stream.

The resulting syn-gas stream is then conducted via line 48 to second acid gas scrubbing zone AGS2 wherein the acid gas CO₂ is removed. Any suitable acid gas treating method can be used in the practice of the present invention as previously described for acid gas scrubbing zone AGS1.

The syn-gas product, which is now substantially free of CO₂, is comprised predominantly of CO, H₂ with small amounts of CH₄ is conducted via line 50 to first stage Fischer-Tropsch ethanol reaction zone containing a suitable catalyst. The catalyst used will be a catalyst, preferably with minor amounts of an alkali metal promoter, capable of the producing C₁ and C₂ alcohols from at least a portion of the syn-gas feedstream. The reaction product from this first stage ethanol reaction zone is passed to heat recovery zone HR4 wherein the temperature of the stream is dropped to the point where a liquid phase and a gaseous phase are formed. This liquid phase and gaseous phase are separated from each other in third separation zone S3. The liquid phase which is comprised primarily of methanol, ethanol with smaller amounts of propanol is sent to Methanol Recovery zone wherein methanol is distilled out and recycled, via line 52, to first stage ethanol reaction zone. An ethanol rich portion is sent via line 54 to ethanol dehydration zone ED wherein a product stream comprised of substantially all ethanol is produced. Ethanol dehydration zone ED also preferably contains an azeotropic distillation section AD which preferably uses hexane to extract water from the system via line 56. A portion of the gaseous phase from third separation zone S3 is sent to a second stage ethanol reaction zone wherein additional ethanol is produced from the gaseous product and another portion is recycled to the first stage of the ethanol reaction zone. The product from the second stage ethanol reaction zone will also be comprised of a liquid phase and a gaseous phase which are separated from each other in fourth separation zone S4. The liquid phase, which will also be comprised primarily of a mixture of low carbon number alcohols is conducted to Methanol Recovery zone. The gaseous phase will be recycle to the second stage of the ethanol reaction zone.

The ethanol-rich stream from ethanol dehydration zone ED is passed via line 58 to ethylene reaction zone ER containing an effective amount of a suitable catalyst, such as an activated alumina, to produce an olefin stream comprised primarily of ethylene with smaller amounts of propylene.

It will be understood that both stages of the ethanol reaction zone are exothermic therefore is may be necessary to remove heat as required, preferably by passing each stream to a heat recovery zone. Any steam produced in such a heat recovery zone can be used in any of the steam reforming zones. The threshold temperature for ethanol production is about 260° C. The ethanol reactor operates at a temperature from about 300° C. to about 500° C., and a pressure from about 650 to about 2,000 psig. The gas hourly space velocity in the ethanol reactor is from about 8,000 to about 50,000 per hour.

Any conventional ethanol producing catalyst can be used in the Fischer-Tropsch reactor of the present invention. Preferred catalysts are those that are based on cobalt with minor amounts of other elements selected from the group consisting of manganese, zinc, chromium and/or aluminum, and an alkali or alkaline earth metal promoter, with potassium carbonate being preferred for economic reasons. The more preferred ethanol catalysts will be comprised of about 65 wt. % to about 75 wt. % cobalt, about 4 wt. % to about 12 wt. % manganese, about 4 wt. % to about 10 wt. % zinc, about 6 wt. % to about 10 wt. % chromium, and/or about 6 wt. % to about 10 wt. % aluminum, wherein all weight percents are based only on the metal content without binder or carrier.

While the catalyst as used consists primarily of the above elements in their elemental form, the catalysts are typically prepared from a mixture of metal salts. Nitrates, carbonates, oxides and sulfides are preferred. The catalysts used in the both ethanol stages and the ethanol reactor of this invention will be subjected to a “conditioning” process wherein the salts are largely reduced to their metallic state, with some oxides remaining to form a lattice structure referred to as spinels. The spinel structure help give the catalysts their overall special structure. The catalysts may be used in their pure (or concentrated) form, or they may be diluted with carbon, by loading onto carbon pellets. The later is often referred to as supported catalyst. A “pure” catalyst will tend to run hotter than a supported catalyst. On the other hand a “pure” catalyst will be more active and hence can be used at lower reaction temperatures. Thus a compromise must often be reached between the desirability of using a more reactive catalyst and the need to dilute it in order to facilitate temperature control.

What is claimed is:

1. A process for generating electrical power from a coal fuel source while producing low carbon number alcohols, which process comprises:
i) introducing a first carbonaceous feedstock and an effective amount of steam into a first reforming zone operated under reforming conditions thereby producing a fuel gas product stream comprised of solids, H₂, CO, CH₄, CO₂ and H₂S, which fuel gas product stream is a high temperature stream;
ii) passing said high temperature fuel gas stream to a heat recovery zone wherein its temperature is reduced to a temperature suitable for a CO-shift conversion reaction zone and wherein at least a portion of the heat of the fuel gas is utilized to generate steam;
iii) passing said fuel gas stream which is now at a lower temperature to a solids recovery zone wherein a substantial amount of the solids of said lower temperature fuel gas stream are removed, thereby resulting in a substantially solids-free lower temperature fuel gas stream;
iv) conducting said substantially solids-free fuel gas stream to a CO shift conversion zone operated at a temperature from about 180° C. to about 280° C. wherein CO is reacted with H₂O in the presence of a shift conversion catalyst to covert at least a portion of the CO and H₂O into CO₂ and H₂, thereby resulting in a substantially solids-free CO-lean fuel gas stream comprised primarily of CO₂, H₂S, CH₄ and H₂;
v) conducting said substantially solids-free fuel gas stream resulting from step iv) to a heat recovery zone wherein the stream is reduced to a temperature effective for conducting to an acid scrubbing zone;
vi) conducting said substantially solids-free fuel gas steam of step v) to an acid gas scrubbing zone wherein at least a portion of the H₂S and CO₂ are removed, thereby resulting in an acid gas rich stream and an acid gas lean fuel gas stream, which substantially acid gas lean fuel gas stream contains at least about 80 vol. % H₂;
vii) conducting said substantially acid gas lean fuel gas stream from said acid gas scrubbing zone to a power plant wherein it is used as fuel to a gas turbine associated with an electrical generator;
viii) conducting said acid gas rich stream to a sulfur removal zone wherein sulfur compounds, including H₂S, are removed thereby resulting in a CO₂-rich stream;
ix) conducting said CO₂-rich stream along with a second carbonaceous feedstock to a second reforming zone operated under reforming conditions including temperatures from about 650° F. to about 1750° F. wherein a syn-gas product stream is produced comprised of solids, H₂, CO, CH₄, and CO₂;
x) passing said syn-gas product stream to a second heat recovery zone wherein its temperature is reduced and wherein at least a portion of the heat of the syn-gas is utilized to generate steam;
xi) passing said syn-gas stream now at a lower temperature to a solids recovery zone wherein a substantial amount of the solids of the solids waste stream are removed thereby resulting in a substantially solids-free lower temperature syn-gas stream; and
xii) passing said substantially solids-free lower temperature syn-gas stream to a second acid gas removal zone wherein substantially all of the CO₂ is removed, thereby resulting in an acid gas rich stream and an acid gas lean syn-gas stream comprised primarily of H₂, CH₄ and CO;
xiii) passing at least a portion of said acid gas lean syn-gas stream to a Fischer-Tropsch reaction unit containing a suitable catalyst for the production of methanol and operated at Fischer-Tropsch reaction conditions, thereby producing a stream containing predominantly methanol;
xiv) passing at least a portion of said methanol and a portion of said lean syn-gas stream of step xiii) above to a Fischer-Tropsch reaction unit containing a suitable catalyst for the production of ethanol and operated at Fischer-Tropsch reaction conditions, thereby producing a stream containing predominantly ethanol; and
xv) conducting at least a portion of the ethanol to a dehydration zone where at least a portion of the ethanol is converted to ethylene.

2. The process of claim 1 wherein the carbonaceous feedstock for said first reforming zone and said second reforming zone is selected from the group consisting of: i) petroleum derived carbonaceous materials; ii) bitumens; iii) natural gas; iv) coal; v) coal derived materials; and vi) biomass.

3. The process of claim 1 wherein the carbonaceous feedstock for said first reforming zone is a coal selected from lignite, sub-bituminous, bituminous and anthracite.

4. The process of claim 1 wherein said first reforming zone is comprised of three temperature zones, each serially and fluidly connected to each other and each at a higher temperature than the previous immediate upstream temperature zone which respect to the flow of feedstock.

5. The process of claim 2 wherein said first reforming zone is comprised of three temperature zones, each serially and fluidly connected to each other and each at a higher temperature than the previous immediate upstream temperature zone which respect to the flow of feedstock.

6. The process of claim 5 wherein the coal is anthracite and said first reforming zone has a fourth temperature zone operated at a higher temperature than the third temperature zone.

7. The process of claim 1 wherein said first and second acid gas scrubbing zones contains an amine solution.

8. The process of claim 5 wherein the amine is selected from the group consisting of diethanol amine, mono-ethanol amine, a mixture thereof.

9. The process of claim 1 wherein the acid gas lean stream of step vi) contains at least about 85 vol. % H₂.

10. The process of claim 1 wherein the acid gas lean stream of step vi) contains at least about 90 vol. % H₂.

11. The process of claim 1 wherein the carbonaceous feedstock to said first reforming zone is a coal and the carbonaceous feedstock to said second reforming zone is a biomass.

12. The process of claim 11 wherein the biomass is a plant biomass.

13. The process of claim 12 wherein the plant biomass is a cellulosic based biomass material.

14. The process of claim 1 wherein said second reforming zone is comprised of three temperature zones, each serially and fluidly connected to each other and each at a higher temperature than the previous immediate upstream temperature zone which respect to the flow of feedstock.

15. The process of claim 1 wherein said second reforming zone is comprised of three temperature zones, each serially and fluidly connected to each other and each at a higher temperature than the previous immediate upstream temperature zone which respect to the flow of feedstock.

16. The process of claim 1 wherein the ratio of steam to carbonaceous feedstock, on a volume to volume ratio is about 0.2 to 2.5.
